(19) United States
(12) Patent Application Publication
(10) Pub. No.: US 2021/0255178 A1
(43) Pub. Date: Aug. 19, 2021

(54) SUBSTRATE WITH CHANNELS FOR CONTROLLED FLUID FLOW IN BIOLOGICAL ASSAY SAMPLING

(71) Applicant: Quidel Corporation, San Diego, CA (US)

(72) Inventors: Samantha CHANG, San Diego, CA (US); Irene Sinn BLANDY, San Diego, CA (US); Robert REED, San Diego, CA (US); Cristina CHARLES, San Diego, CA (US); Lauren HEDGES, San Diego, CA (US)

(21) Appl. No.: 17/144,991

(22) Filed: Jan. 8, 2021

Related U.S. Application Data
(60) Provisional application No. 62/959,748, filed on Jan. 10, 2020.

(51) Int. Cl. G01N 33/543

(52) U.S. Cl. CPC G01N 33/54366

(57) ABSTRACT

Immunoassay devices with plurality of fluid flow channels that are discrete and designed for optimal fluid control are described. Substrates configured to control the rate of fluid flow for in-situ immunoassay measurements to detect and quantify the presence of one or more analytes of interest in a sample are also described. More specifically, the present disclosure relates to consumables for lateral flow assays, which in conjunction with an instrument detect markers or causative agents of medical conditions.

### CROSS-REFERENCE TO RELATED APPLICATIONS

This application claims the benefit of U.S. Provisional Application No. 62/959,748, filed Jan. 10, 2020 which is incorporated herein by reference in its entirety.

### TECHNICAL FIELD

The subject matter described herein relates to substrates configured to control the rate of fluid flow for in-situ immunoassay measurements to detect and quantify the presence of one or more analytes of interest in a sample. More specifically, the present disclosure relates to consumables for lateral flow assays, which in conjunction with an instrument detect markers or causative agents of medical conditions.

### BACKGROUND

Lateral flow assays are an established technology that can be adapted for a variety of testing applications for sensors, diagnostics, and indicators. Lateral flow assays typically consist of a material or substrate to transport a fluid sample of interest from the point of application (e.g., the sample collection zone) to the detection zone(s) via passive capillary action. For example, rapid lateral flow immunoassay test devices are used in both clinical and home settings. These devices are used to test for a variety of analytes, such as hormones, proteins, urine, or plasma components and the like. These devices generally include a lateral flow test strip, such as nitrocellulose or filter paper, a sample application area, test results area, and an analyte specific binding reagent that is bound to some kind of detectable “label” or “reporter,” such as a colored particle (such as a europium bead), a fluorescent or luminescent tag, or an enzyme detection system. The simplicity of such devices is a factor in maintaining their use in the marketplace. Because the method of fluid transport is passive, the rate of flow as well as the specific flow path is largely fixed by the viscosity of the liquid sample, the substrate material, and the chemical nature of any coatings that may be applied (e.g., hydrophilic or hydrophobic). It would be advantageous to alter the flow rate or control the uniformity of fluid flow without adding extra components or materials to the substrate. An approach to modify and regulate the flow rate and flow uniformity of a fluid sample deposited on a substrate in a lateral flow assay is desired.

### BRIEF DESCRIPTION OF THE FIGURES

FIG. 1 illustrates an architecture including a remote server, a database, and an image-capturing device to collect an image from a test strip in an enclosure, according to some embodiments.

FIGS. 2A-2C illustrate an immunoassay device (FIG. 2A) and fluid progression along the device (FIGS. 2B-2C).

FIGS. 3A-3C illustrate an immunoassay device (FIG. 3A) and its substrate with a plurality of fluid flow channels (FIGS. 3B-3C), according to some embodiments.

FIGS. 4A-4B illustrate an immunoassay device (FIG. 4A) and its substrate with a plurality of fluid flow channels (FIG. 4B), according to some embodiments.

FIGS. 5A-5B illustrate a test strip with a plurality of fluid flow channels and dimensions of certain of its features, according to some embodiments.

FIGS. 6A-6B show data of fluid flow on test strips with and without a diamond-shaped fluid control feature at the channel entrance, and the change in flow rate depending on whether a user places a fluid sample fast or slow onto the sample zone of the test strip.

FIGS. 7A-7B illustrate test strips having conjugate zones and/or capture zones comprised of an array of reagent drops, according to some embodiments.

FIGS. 8A-8B are images of a test strip with a plurality of individual, discrete fluid flow channels (FIG. 8A) and one with a plurality of fluid flow channels that are not discrete (FIG. 8B) after testing for fluid flow and signal capture.

FIGS. 9A-9B illustrate a test strip with a barrier region and a test strip without a barrier region, according to some embodiments.

FIG. 9C shows results from testing fluid flow on the test strips in FIGS. 9A-9B, according to some embodiments.

FIGS. 10A-10B illustrate alternative patterns for creating a flow channels and flow on a test strip.

FIG. 11 is a flowchart illustrating steps in a method for diagnosing, treating, or both, a condition or disorder in a subject, according to some embodiments.

FIG. 12 is a block diagram illustrating an example computer system with which the client and server of FIG. 1, and the method of FIG. 11 can be implemented, according to some embodiments.

### DETAILED DESCRIPTION

### Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

“Sample” is any material to be tested for the presence or amount of an analyte of interest. Preferably, a sample is a fluid sample, preferably a liquid sample. Examples of liquid samples that may be tested using a test device include bodily fluids including blood, serum, plasma, saliva, urine, ocular fluid, semen, sputum, nasal discharge, and spinal fluid.

### General Overview

Embodiments consistent with the present disclosure take advantage of manufacturing techniques for modifying nitrocellulose strips into multiple channels with desired fluid flow properties. In some embodiments, the nitrocellulose strips provide a substrate for simple yet accurate diagnostic procedures for selected diseases (e.g., legionella, influenza, Ebola, Lyme disease, and the like). The types of tests consistent with embodiments in the present disclosure may include any type of spectroscopic analysis of test assays using electromagnetic radiation, such as, without limitation, absorption spectroscopy (ultra-violet, visible, or infrared) including reflectance or transmittance spectroscopy, or emission spectroscopy, including fluorescence and luminescence spectroscopy, Raman spectroscopy, and any type of radiation scattering. Moreover, embodiments as disclosed herein may further exploit the networking capabilities of such appliances to enhance the processing, cataloging, regulating, and cross-referencing capabilities of each test by using cloud-computing solutions. Accordingly, in some embodiments, a high quality (e.g., high spatial and spectral resolution) image, sequence of images, a video, or a processed version of them is uploaded to a remote server that can perform massively parallel computations to provide, in a reduced time, a diagnostic result. Such analyzed material may be processed immediately, at a later date/time and/or may be compared to previously collected materials to determine differences over time, e.g., a time evolution of the analyte across a test strip. Such analyzed material may also, after user de-identification, be used for analyses in the interest of public health, or to provide additional benefits to the user of the test by cross-referencing the results to others with specific criteria, e.g., age group, gender, geographic location, pathogen characteristics, and the like.

The subject system provides several advantages, including the ability for a user to quickly learn whether a disease is present or latent, or mild or severe, without the need to access specialized personnel, or a complex machine or instrument.

Although many examples provided herein describe a user's personal information and data as being identifiable, or a download and storage of a user interaction history with one or more remote clinics, each user may grant explicit permission for such user information to be shared or stored. The explicit permission may be granted using privacy controls integrated into the disclosed system. Each user may be provided notice that such user information will be shared with explicit consent, and each user may at any time end the information sharing, and may delete any stored user information. Further, in some embodiments, the stored user information may be encrypted to protect user security and identity.

### Example System Architecture

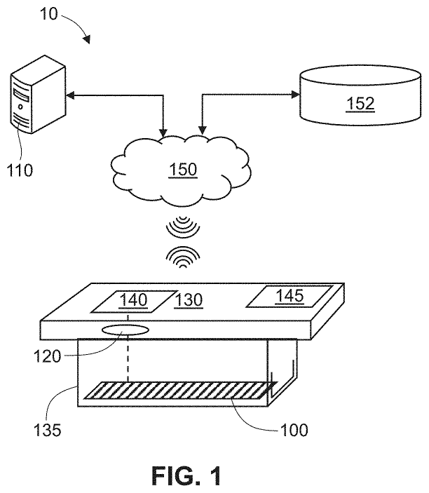

FIG. 1 illustrates an architecture 10 including a remote server 110, a database 152, and an image-capturing device 130 to collect an image from a test strip 100 in an enclosure 135, according to some embodiments. In architecture 10, test strip 100 and enclosure 135 may be consumables that the user may dispose of after use. For example, test strip 100 may be replaced after each use of a test sample, while enclosure 135 may be used a few more times. In that regard, test strip 100 and the enclosure may be part of a package requested by the user to a clinical service provider. The package may include one enclosure 135 and multiple test strips 100 that may be used with it. In some embodiments, enclosure 135 may be a semi-permanent or permanent auxiliary box that can be used multiple times (e.g., a cassette or cartridge), independently of whether they are part of the package. In some embodiments, enclosure 135 is a housing or cartridge to ease the handling of test strip 100. In other embodiments, test strip 100 is an immunoassay test strip, such as a dip stick. That is, enclosure 135 is optional, and if present, can be a flexible laminate, such as that disclosed in U.S. Patent Application Publication No. 2009/02263854 and shown in Design Patent No. D606664.

In addition to the consumables, image-capturing device 130 may include a smartphone or other mobile computing device (e.g., tablet, pad, or even laptop) provided by the user. Image-capturing device 130 may generally include a sensor array 140 and an optics coupling mechanism 120 (e.g., a lens system with autofocus capabilities). Image-capturing device 130 may also be configured to couple wirelessly, through a network 150, with a remote server 110 and a remote database 152. Remote server 110 may provide support for an image-capturing application 145 installed in image-capturing device 130. The support may include installation, update, and maintenance of image-capturing application 145, retrieval of raw data (e.g., pictures, sequences of pictures and videos) for storage in database 152, image processing, and the like.

While some of the descriptions herein are focused on fluorescence spectroscopic analysis of the test strip, some embodiments consistent with the present disclosure may include any other type of electromagnetic interaction and spectroscopic analysis. Some examples of spectroscopic analysis consistent with the present disclosure may include Raman spectroscopy, infrared absorption spectroscopy, infrared reflectance/transmittance spectroscopy, and the like. Furthermore, in some embodiments, the light emitting source may be replaced by an optical coupling mechanism (e.g., a lens, mirror, prism, diffraction grating, or any combination thereof) to use solar radiation (e.g., during day light) or any exterior illumination to excite a spectroscopic response of the area of interest in the test strip.

Enclosure 135 is configured to avoid or control any external light to interfere with the fluorescence excitation light or with the fluorescence emission light collected by the image-capturing device. For example, it is desirable to illuminate the area of interest in the test strip uniformly (e.g., no shadows, bright spots, or other artifacts) to create a smooth spectroscopic background that can be filtered out by the image-capturing application in the image-capturing device.

Some embodiments extract a value for assessing a diagnostic of the assay by spatially and/or spectrally filtering an image of test strip 100. Accordingly, filtered pixel values may be aggregated and compared to a pre-selected threshold. Thus, when the aggregated value is lower or greater than the threshold, a disease diagnosis may be positive. Some embodiments may include error values based on statistical analysis and calibration, to provide a confidence interval for the diagnostics. In other embodiments, the information can be compared between the area that one analyte band takes with a similar area where no capture of the fluorescent complex exists.

### Substrate Including a Fluid Channel and a Fluid Control Feature

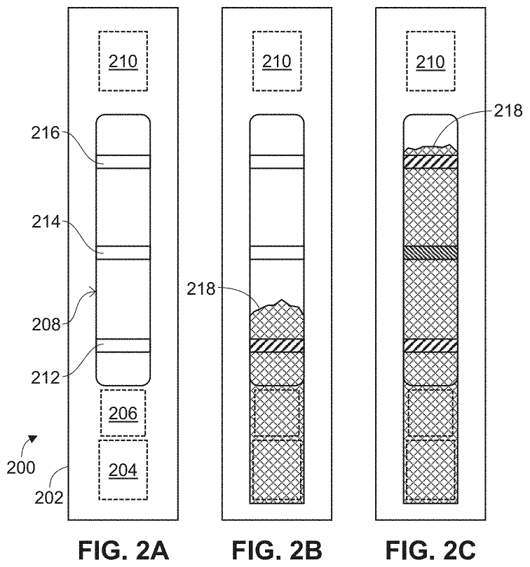

FIGS. 2A-2C illustrate progression of a sample upon placement on an assay device. The sample may comprise a fluid composed of a reagent or processing solution, if needed, and a sample of interest (e.g., from a patient, a test container, and the like). The reagent or processing solution is optional, and may be useful for to facilitate flow of a sample through the different portions of an immunoassay device in a “lateral direction” (e.g., from left to right in the figures) by capillary action. The sample of interest may be collected from the patient via a cotton swab (e.g., a nasal swab, or any other bodily cavities), a syringe or a scoop, in a pre-selected volume, e.g., 100 microliters (μL), or more.

With initial reference to FIG. 1A, an immunoassay device 200 includes a substrate 202. Deposited on or formed in the substrate are a sample zone 204 in fluid communication with a conjugate zone 206, a capture zone 208, and an optional absorbent pad 210. Typically, conjugate zone 206 is downstream from sample pad 204; capture zone 208 is downstream from conjugate zone 206; and absorbent pad 210 is downstream from capture zone 208. In some embodiments, an image-capturing device is configured to capture and process an image of at least a portion of capture zone 208 (e.g., image-capturing device 130, cf. FIG. 1). Accordingly, a light source may be configured to excite a signal, such as fluorescence light, from the test strip. In some embodiments, the emitted signal, such as fluorescence light, has a wavelength within the selected color in a sensor array in the image-capturing device.

In some embodiments, conjugate zone 206 includes a mobilizable, detectable species. Examples of mobilizable, detectable species are known in the art and depend on the analyte of interest (e.g., an infectious agent, or chemical components such as a drug or a contaminant). In some embodiments, the immunoassay device lacks a conjugate zone, and the mobilizable, detectable species is provided, for example as a lyophilized material, in a container with the immunoassay device. The sample and the lyophilized material are mixed, and the mixture is deposited onto sample pad 204.

In some embodiments, capture zone 208 includes one or more lines, bands, or spots such as a first control zone 212, a first test zone 214, and a second test zone 216 (hereinafter, collectively referred to as “capture zones”). Accordingly, the shape and number of capture zones may include multiple varieties: dots, drops, lines, and arrays of dots and/or lines, and even curved shapes having more complex form factors. Capture zones include at least one immobilizable species that has chemical or physical affinity to at least a portion of the conjugate complexes formed between a mobilizable, detectable species and the analyte of interest or a control analyte. The binding species in each capture zone may be deposited or printed from a solution and allowed to dry for a period of time (e.g., a few minutes, hours, or overnight). In some embodiments, each control or test line in the capture zone comprises a binding member for a particular analyte, and each analyte binds to a distinct mobilizable, detectable species, where the detectable species differ in signal emission, e.g., wavelength or type. In one embodiment, each control or test line binds a conjugate of an analyte and a mobilizable, detectable species that generates an optical signal at a different wavelength. Exemplary immunoassay test strips are described, for example, in U.S. Pat. Nos. 9,207,181, 9,989,466, and 10,168,329 and in U.S. Publication Nos. 2017/0059566 and 2018/0229232, each of which is incorporated by reference herein.

Immunoassay device 200 may be configured uniquely for detection of a particular pathogen or analyte of species of interest. These include, but are not limited to, proteins, haptens, immunoglobulins, enzymes, hormones, polynucleotides, steroids, lipoproteins, drugs, bacterial antigens, and viral antigens. With regard to bacterial and viral antigens, more generally referred to in the art as infections antigens, analytes of interest include Streptococcus, Influenza A, Influenza B, respiratory syncytial virus (RSV), hepatitis A, B, and/or C, pneumococcal, human metapneumovirus, and other infectious agents well known to those in the art. In some embodiments, a test device is intended for detection of one or more antigens associated with Lyme disease. In some embodiments, an immunoassay device is intended for use in the field of women's health. For example, test devices for detection of one or more of fetal-fibronectin, chlamydia, human chorionic gonadotropin (HCG), hyperglycosylated chorionic gonadotropin, human papillomavirus (HPV), and the like, are contemplated. In another embodiment, an immunoassay device is configured for detection of vitamin D, and is designed for interaction with the apparatus and method of normalization described herein. The techniques used to measure a signal from the immunoassay device may include any immunoassay technique such as non-competitive assay techniques, competitive assay techniques (e.g., homogeneous competitive assay, inhomogeneous competitive assay), and the like.

In some embodiments, the analyte of interest may include a disease carrying pathogen such as respiratory syncytial virus (RSV), Flu A virus, Flu B virus, or human metapneumovirus (hMPV). In some embodiments, the analyte of interest may include a controlled substance, such as drugs and other illegal or proscribed substances (e.g., steroids and the like). For example, some embodiments may include detection and measurement of drugs such as fentanyl, buprenorphine, oxycodone, and/or 7-aminoclonazepam.

With continued reference to FIG. 2A, sample pad 204 receives a sample suspected of containing the analyte of interest. Conjugate zone 206, in some embodiments, contains two dried conjugates that include particles containing a detectable label element, such as a fluorescent element. An exemplary fluorescent element is a lanthanide material, such as one of the fifteen elements lanthanum, cerium, praseodymium, neodymium, promethium, samarium, europium, gadolinium, terbium, dysprosium, holmium, erbium, ytterbium, lutetium, and yttrium. In one embodiment, the lanthanide material is embedded in or on a particle, such as a polystyrene particle. The particles can be microparticles (particles less than about 1,000 micrometers in diameter, in some instances less than about 500 micrometers in diameter, in some instances less than 200, 150, or 100 micrometers in diameter) containing a luminescent or fluorescent lanthanide, wherein in some embodiments, the lanthanide is europium. In some embodiments, the lanthanide is a chelated europium. The microparticles, in some embodiments, have a core of a lanthanide material with a polymeric coating, such as a Europium core with polystyrene coating. A binding partner for the analyte(s) of interest in the sample is/are attached to or associated with the outer surface of the microparticles. In some embodiments, the binding partner for the analyte(s) of interest is an antibody, a monoclonal antibody or a polyclonal antibody. A skilled artisan will appreciate that other binding partners can be selected, and can include complexes such as a biotin and streptavidin complex. Upon entering conjugate zone 206, the liquid sample hydrates, suspends, and mobilizes the dried microparticle-antibody conjugates and carries the conjugates together with the sample downstream on the test strip to the control or reference and/or test lines in the capture zone 208. As the sample and microparticle-antibody conjugates continue to flow downstream on a test strip, if the analyte of interest is present in the sample, the fluorescent microparticle-antibody conjugate, which is now bound with an antigen/analyte of interest, will bind to the specific binding member for the analyte of interest that is immobilized at a conjugate line in the conjugate zone 208. In some embodiments, a single test line is present on immunoassay substrate. In some embodiments, at least two or more test lines are present. By way of example, capture zone 208 may be designed for detection and/or discrimination of influenza A and influenza B and can include a first test line 214 to detect influenza A and a second test line 216 to detect influenza B. Microparticle-antibody conjugates including microparticles coated with antibodies specific for influenza A and microparticles coated with antibodies specific for influenza B may be included in conjugate zone 206, and in some embodiments, downstream of a control line 212. The first test line for influenza A includes a monoclonal or polyclonal antibody to a determinant on the nucleoprotein of influenza A and the second test line for influenza B includes a monoclonal or polyclonal antibody to a determinant on the nucleoprotein of influenza B. If an antigen is present in the sample, a typical immunoassay sandwich will form on the respective test line that matches the antigen in the sample.

The immunoassay test device is intended for receiving a wide variety of samples, including biological samples from human bodily fluids, including but not limited to nasal secretions, nasopharyngeal secretions, saliva, mucous, urine, vaginal secretions, fecal samples, blood, etc. The kit described herein, in some embodiments, is provided with a positive control swab or sample. In some embodiments, a negative control swab or sample is provided. For assays requiring an external positive and/or negative control, the user may be prompted to insert or apply a positive or negative control sample or swab.

An immunoassay band emits fluorescence light primarily from fluorophores bound to the target analyte, as they are fixed on the substrate by adherence to the immuno-proteins in the immunoassay strip (e.g., adsorption, chemi-sorption, immune-ligand, and the like). Accordingly, the presence of a red emission within the boundaries of the band is mostly attributable to the presence of the target analyte (e.g., presence of pathogenic antigens, and the like). However, the amount of red signal within the boundaries of the immunoassay band may include some background. To better assess the background signal (e.g., not originated by target analytes bound to the antibodies on the band), some test strips may include a blank control area.

In some embodiments, first and second control lines are disposed on either side of a test line relative to the flow direction. Accordingly, the first and second control lines provide a start/end signal for the assay. This may anticipate a negative result, even when only a portion of the second control line becomes wetted with sample. In some embodiments, the image-capturing device may capture a pixelated image of capture zone 208, and therefore the progression of a fluid front may be captured in time as it flows from sample zone downstream on the test substrate. Accordingly, some embodiments may provide metrics and performance data for the substrate as sample fluid progression is tracked. This concept is illustrated with respect to FIGS. 2B-2C, where a moving fluid front 218, is illustrated. Time for the fluid front to move between two points on the test strip can be ascertained by, for example, analyzing image frames collected during sample flow between the two points. In addition to ascertaining fluid flow rate, a shape of fluid front 210 (e.g., curvature and tilt) can be visualized in the images, as a metric for the test strip.

The test device shown in FIG. 2B illustrates where a sample has been deposited on sample pad 204 and flowed across the conjugate pad 206 and into the capture zone 208. In the device of FIG. 2C, the sample has progressed through capture zone 208. In some embodiments, it is desirable to make a measurement on the sample after fluid front 210 reaches a certain landmark position. Accordingly, in some embodiments, the full progression of fluid front 210 along substrate may be recorded by an image-capturing device. In some embodiments, it may be desirable that fluid front 210 forms a line substantially perpendicular to the progression of the sample, to ensure that substantially an entire width of a test line in the capture zone is contacted by the sample at approximately the same time. In some embodiments, fluid front 210 may have any shape (concave, convex, irregular, and the like), and the image-capturing device may be configured to follow fluid front 210 as it progresses across the capture zone. The time for sample to flow from the sample zone to the end of the capture zone may be 5 minutes, 7 minutes, 10 minutes, 12 minutes, 15 minutes, 17 minutes, 20 minutes, or more.

The substrate of the immunoassay device may be a laminate comprised of a first base or support layer and a second membrane layer, where the first support layer can be hydrophobic or hydrophilic and the second membrane layer is adhered to the first layer, the second membrane layer being bibulous in nature and/or capable of capillary flow. The support layer may be hydrophobic or impermeable, such as polyethylene terephthalate, polyesters, silicone, and the like.

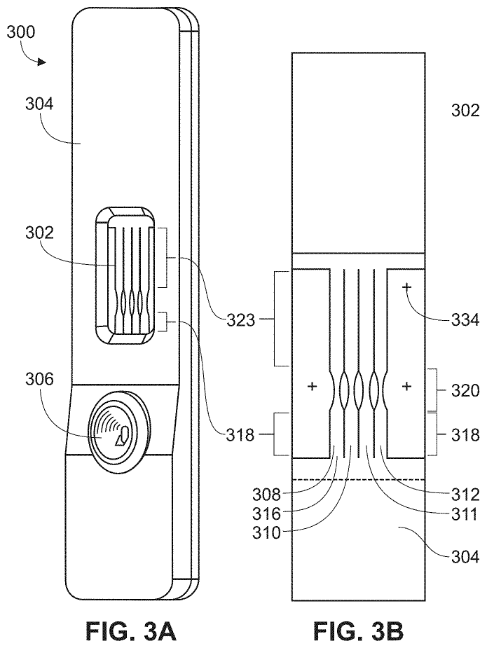

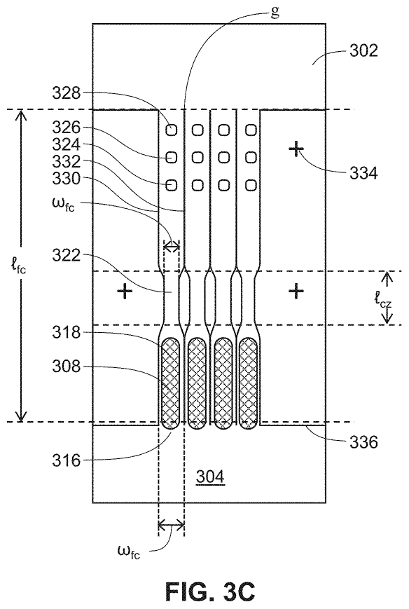

With the introduction provided in FIGS. 2A-2C of a general immunoassay device, immunoassay devices of the present disclosure will now be described. In a first aspect, an immunoassay device is comprised of a single, unitary substrate comprising a plurality of discrete fluid flow channels. The substrate may be a laminate material, as described above, and is a single, unitary laminate substrate configured as will now be described with reference to FIGS. 3A-3C. FIG. 3A illustrates an immunoassay device 300 comprised of a test strip 302 inserted into an optional housing 304. FIG. 3B illustrates test strip 302. Test strip 302 comprises a single sample zone 304, which is accessible via port 306 in the optional housing of FIG. 3A. Single sample zone 304 is common to and in fluid communication with a plurality of fluid flow channels, such as the individual, discrete channels identified as 308, 310, 312, and 314. Each fluid flow channel is in direct or indirect fluid communication with the sample zone at a channel entrance region of each fluid flow channel, such a channel entrance 316 of channel 308.

Each fluid flow channel in the plurality of channels has a length l_{fc} and a width w_{fc}. Each fluid flow channel comprises a capture zone downstream from the channel entrance region and a channel constriction zone positioned between the channel entrance region and the capture zone, the channel constriction zone having a width w_{cz} and a length l_{cz}, the channel constriction zone width w_{cz} corresponding to a value in the range determined by (i) a minimum value that is equal to or greater than a diameter of a particulate reagent deposited on or to be deposited on the substrate or (i′) a minimum value that is equal to or greater than about 25% of the fluid flow channel width w_{fc}, and (ii) a maximum value that is equal to or less than about 75% of the fluid flow channel width w_{fc}.

With reference to FIGS. 3B-3C, fluid flow channel 308 has a width w_{fc}and a length l_{fc}. Fluid flow channel also comprises a capture zone 318 positioned downstream from the channel entrance region 316 and upstream from a channel constriction zone, indicated in FIG. 3B generally across the plurality of fluid flow channels as 320 and in FIG. 3C as constriction 322 in fluid flow channel 308. The constriction in each fluid flow channel, such as constriction 322, has a width w_{cz} and a length l_{cz}. In embodiments where the width of the constriction varies along its length, width w_{cz} corresponds to the smallest width along the length l_{cz}. Downstream of the channel constriction zone in each fluid flow channel is a capture zone 323 comprising one or more capture or test or control “lines”. Use of the word “line” is not intended to impart a geometric shape of a line, as the line can be any geometric shape, such as a circle, diamond, triangle, or an array of any geometric shape(s). In fluid flow channel 308 of FIG. 3C, downstream of constriction 322 are three capture lines, 324, 326, 328.

Test strip 302 of FIGS. 3B-3C is a unitary substrate—that is, the substrate is a single continuous material. In an embodiment, the single continuous substrate is a laminate of a support material and a membrane material. The membrane material is treated to form the fluid flow channels, including the constriction region and the capture lines, as will now be described. In one embodiment, the membrane material is a bibulous material, such as nitrocellulose. The nitrocellulose is exposed to chemicals or lasers to remove or etch away portions of the nitrocellulose to form the flow channels. For example, fluid flow channel 308 in FIGS. 3B-3C is formed by etching away membrane material to form opposing side walls 330, 332. The dimensions of the side walls are adjusted in the constriction region, as can be appreciated. The test strip additionally may include fiducials, such as fiducial 334, and transverse barriers, such as barrier 336. The fiducial assists in optical analysis of the conjugate lines and the barriers assist in fluid flow control on the test strip. In one particular embodiment, a laser is used to ablate substrate membrane material in a controlled fashion. Laser ablation generally refers to a process for removing a material using incident light of a certain wavelength. In polymeric materials, for instance, the incident light generally induces photochemical changes in the polymer that results in chemical dissolution. Any known laser may be employed in the present invention, including, for instance, CO₂ lasers, pulsed light lasers, diode lasers, ND:Yag 1064 nm and 532 nm lasers, alexandrite and Q-switched lasers, pulsed dye lasers, optical and RF lasers, erbium lasers, ruby lasers, and holmium lasers. In a preferred embodiment, a CO₂ laser is used to etch a nitrocellulose membrane that is mounted on a supporting fixture. Through use of a moving beam or an x-y table, precision channels are created on the nitrocellulose to define, for example, fluid flow channels and other fluid features. In addition, other optical devices may be employed in conjunction with the laser to enhance the channel formation, such as optical lenses, mirrors, and the like. In another embodiment, a Nd:YVO4 solid-state laser having picosecond pulses is used, for example at a 532 nanometer wavelength and a 12 picosecond pulse length, a 10 micro-joule pulse energy and a 10 kilohertz pulse frequency, with a beam focused on substrate 301 using a 100 millimeter F-theta lens and a feed rate of 25 milliseconds per second. The parameters for laser ablation of the substrate, such as wavelength, pulse duration, pulse repetition rate, and beam quality, for any given laser can be determined by a skilled artisan.

With reference to FIG. 3C, each fluid flow channel is physically separated from an adjacent fluid flow channel by a gap, g, corresponding to a region of ablated substrate or corresponding to thickness/width of a side wall. In some embodiments, g may have dimensions of at least about 0.01 mm, 0.025 mm, 0.03 mm, 0.05 mm, 0.07 mm, 0.08 mm, 0.09 mm, 0.1 mm, 0.2 mm, 0.3 mm, 0.4 mm, 0.5 mm, 0.6 mm, 0.7 mm, 0.8 mm, 0.9 mm, or 1 mm, or is between about any two of these discrete values.

In some embodiments, the width, w_{fc}, of each fluid channel may have dimensions of about 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, or 2 mm, or is between about any two of these discrete values. For example, in some embodiments, w_{fc} may be 1 mm, and w2 may be 0.5 mm.

In some embodiments, a capture line or dot is disposed on the substrate using ink-jet techniques such as in the printing industry. As mentioned above, in some embodiments, the capture line can comprise an array of dots where the array has dimensions of m×n (e.g., columns×rows). Accordingly, in some embodiments, the capture zone on the test strip and/or the capture line in an individual fluid flow channel may include an m×n array of discrete drops or dots, where m and n are greater than or equal to one (1), and wherein each dot in the m×n array is separated from an adjacent dot by a distance, x (e.g., “pitch,” or “spacing”). In some embodiments, m and n may be any integer, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more. In some embodiments, the pitch, x, may be between about 20-1000 μm, or between about 50-500 μm, or between about 75-500 μm, or between about 100-500 μm, or between about 150-500 μm, or between about 150-300 μm, or between about 150-250 μm, or between about 200-500 μm. In some embodiments, the volume of formulation deposited on the substrate to form each dot 325 may be between about 20-1000 pL, or between about 50-800 pL, or between about 75-800 pL, or between about 100-600 pL, or between about 150-550 pL, or between about 200-500 pL, or between about 200-450 pL.

In some embodiments, each dot in the array may include a different immobilizable, detectable species, arranged in the m×n array to optimize a capture/detection efficiency, and also to provide and improve a measurement quantification. For example, in some embodiments, dots along the same fluid channel 308 (e.g., along the same column in the m×n array) may include the same immobilizable, detectable species. Accordingly, as the sample fluid progresses through the column of dots in the fluid flow channel, a decay in the signal collected from the dots along each fluid channel may be mathematically fitted to a model according to a precise value of the analyte concentration in the sample (given a known concentration of the detectable species on dots). In some embodiments, the dots along the same row may include the same, immobilizable, detectable species, while each row is associated with a different species. Accordingly, multiple analytes of interest may be detected along each of fluid flow channels, and the plurality of channels enables a statistical comparison of the measurements.

In some embodiments, conjugates specific to each assay (e.g., RSV, Flu A, Flu B, hMPV, and the like) are printed onto capture lines in conjugate zone for each fluid flow channel separately, thus providing a test assay for multi-analyte detection. Each drop in the array is deposited onto the substrate from a precision liquid dispensing instrument. In some embodiments, the dispensing instrument permits a user to select droplet volume, drop pitch, and other variables. A user can also select whether multiple droplets are deposited at each position in the array in a single pass of the instrument dispensing head or in multiple passes of the instrument dispensing head. Different instrument variables may be adjusted to obtain a desired positional accuracy of each dot in the arrays. Some of the instrument variables may include selecting a number of droplets of formulation including the conjugate to the mobilizable, detectable species dispensed to form each drop. In some embodiments, each drop of the array may be formed by dispensing 1, 5, 10, or more droplets of formulation. The droplet volume may be adjusted accordingly, e.g., by tens or hundreds of pico-liters (pL) per each droplet, as desired. Further, in some embodiments, the multiple dots in the array may be deposited on several passes of an injection head. For example, a first number of droplets may be deposited on each dot in a first pass, and a second number of droplets may be deposited on each dot in a second pass. In some embodiments, depositing fewer droplets with multiple passes of the dispensing head improves the positional accuracy of the drops in the array, and more uniform pitch (horizontal and vertical).

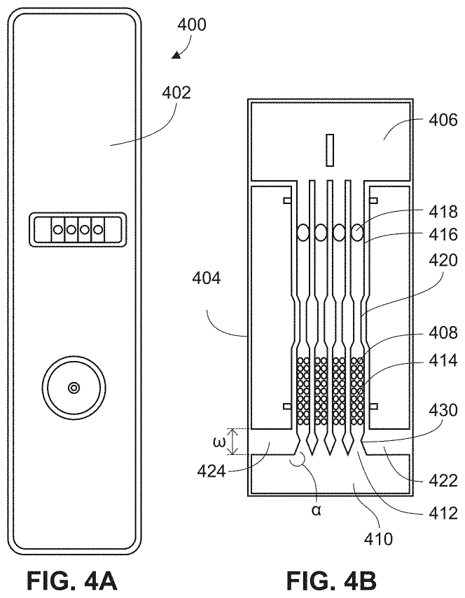

FIGS. 4A-4B illustrate another exemplary immunoassay test device 400. Immunoassay device 400 comprises an optional housing 402 for a test strip 404 which is shown separate from a housing in FIG. 4B. Test strip 404 comprises a single, unitary substrate 406. As mentioned above, substrate 406 can be a laminate material comprised of a support layer and a bibulous layer. The bibulous layer is treated or processed to comprise a plurality of discrete fluid flow channels. The embodiment shown in FIGS. 4A-4B comprises four discrete fluid flow channels, channel 408 being representative. The substrate also comprises a single sample zone or region 410 in direct fluid communication with each channel in the plurality of channels. More specifically, a channel entrance region of each channel, such as channel entrance region 412 of representative channel 408, is in direct fluid communication with no intervening structures, materials, zones and/or components with the sample zone that receives a sample for analysis. Each fluid flow channel in the plurality of channels comprises a conjugate zone, indicated at 414 in representative channel 408. Each fluid flow channel in the plurality of channels comprises a capture zone, indicated at 416 in representative channel 408. The capture zone comprises one or more test lines, such as line 418, with immobilized reagent, as discussed above.

Each fluid flow channel in the plurality of channels also comprises a fluid control zone 420, sometimes referred to as a constriction zone in embodiments where the fluid control zone is designed to slow or restrict fluid flow. The fluid control zone 420 is positioned between the channel entrance and the conjugate zone. In the embodiment of FIG. 4B, the fluid control zone is downstream of the conjugate zone, however, it could also be upstream of the conjugate zone.

Test strip 404 is also configured to include features to guide and/or meter fluid sample placed on the sample zone into the fluid flow channels. Such a feature is the fluid barriers 422, 424. The dimensions of each barrier and the position of each barrier can be adjusted to guide and/or meter fluid into each fluid flow channel. The width of the barrier, indicated as w, and its angle α, can be varied and selected to adjust fluid dynamics. In an embodiment, the barrier is dimensioned to guide and meter a volume of fluid sample placed in the sample zone into each fluid flow channel to achieve a substantially uniform flow rate in each channel in the plurality of channels (e.g., a flow rate that varies across the plurality of channels of less than about 15%, 10% or 5%) and/or containment of the volume of fluid sample in the sample zone and in the fluid flow channels (and ultimately in any absorbent zone at the terminal end of the channels). That is, none of the fluid sample overflows or seeps or spills over into the region identified by 426 in FIG. 4B.

The channel entrance region of each individual fluid flow channel may be dimensioned and configured to regulate and control entrance of a portion of the fluid sample into each channel. In some embodiments, the channel entrance region comprises a fluid control feature, such as a constriction zone 430. The fluid control feature can have any shape desired, such as a quadrilateral shape (e.g., a rhomboidal shape), an hour glass shape, or a diamond shape.

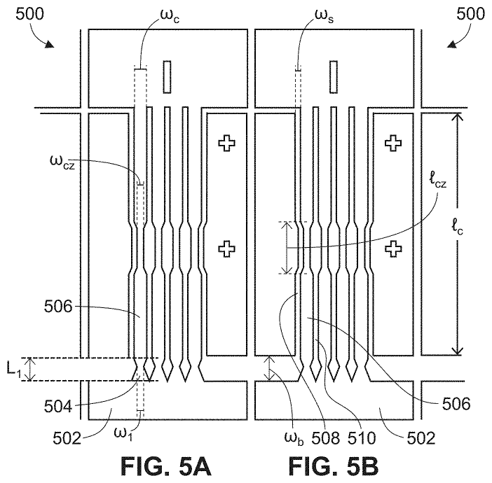

The fluid control zone, or if designed to slow fluid flow, the channel constriction zone, is dimensioned to achieve control of fluid in each fluid flow channel, as will now be discussed with reference to FIGS. 5A-5B. A test strip 500 is shown, where the test strip is comprised of a unitary, continuous piece of material, the material treated as described herein to comprise a plurality of fluid flow channels. Each fluid flow channel has a length lc (FIG. 5B) and a width w_{c} (FIG. 5A). The channel constriction zone in each fluid flow channel has a length lcz (FIG. 5B) and a width w_{cz} (FIG. 5A). In one embodiment, width w_{cz} is a value in a range that has a minimum value and a maximum value. The minimum value of the range is, in one embodiment, equal to or greater than a diameter of a particulate reagent deposited on or to be deposited on the substrate. For example, as discussed above, the conjugate zone in each fluid flow channel may comprise mobilizible detectable particles. The width w_{cz} of the constriction zone is dimensioned to allow the mobilizible detectable particles to flow through the constriction zone. For example, polymeric particles with a detectable label that are used as a reagent in the device have an outer diameter. In embodiments where the immunoassay device is configured with a conjugate zone upstream of the constriction zone that comprises mobilizible, optically detectable solid particles with a certain diameter or when the immunoassay device is designed to include a reagent with a certain dimension that must flow through the constriction zone, the minimal width of the constriction zone is equal to or greater than the dimension of the reagent that must flow there through. Exemplary minimal widths of the constriction zone range from between about 0.01-750 micrometers (0.00001-0.75 mm), 0.01-500 micrometers, 0.01-250 micrometers, 0.01-100 micrometers, 0.01-50 micrometers, 0.01-25 micrometers, 0.01-10 micrometers, 0.01-5 micrometers, 0.01-2 micrometers, 0.01-1.5 micrometers, 0.01-1.0 micrometers, 0.05-750 micrometers, 0.05-500 micrometers, 0.05-250 micrometers, 0.05-100 micrometers, 0.05-50 micrometers, 0.05-25 micrometers, 0.05-10 micrometers, 0.05-5 micrometers, 0.05-2 micrometers, 0.05-1.5 micrometers, 0.05-1.0 micrometers, 0.075-500 micrometers, 0.075-250 micrometers, 0.075-100 micrometers, 0.075-50 micrometers, 0.075-25 micrometers, 0.075-10 micrometers, 0.075-5 micrometers, 0.075-2 micrometers, 0.075-1.5 micrometers, or 0.075-1.0 micrometers.

Alternatively, a minimum value of the dimensional range for the width of the fluid flow channel in the constriction zone is equal to or greater than about 25% of the fluid flow channel width, w_{c}. FIG. 5A shows the width of the fluid flow channel w_{c} and the width of the channel in the constriction zone, w_{cz}. According to this embodiment, w_{cz} equals 0.25 times w_{c}. In other embodiments, w_{cz} is equal to or greater than 0.10 times w_{c}, w_{cz} is equal to or greater than 0.15 times w_{c}, w_{cz} is equal to or greater than 0.20 times w_{c}, w_{cz} is equal to or greater than 0.30 times w_{c}, w_{cz} is equal to or greater than 0.35 times w_{c}, w_{cz} is equal to or greater than 0.40 times w_{c}, w_{cz} is equal to or greater than 0.50 times w_{c}. In other embodiments, w_{cz} equals between about 0.05-0.75 times w_{c}, w_{cz} equals between about 0.1-0.75 times w_{c}, w_{cz} equals between about 0.1-0.5 times w_{c}, w_{cz} equals between about 0.15-0.5 times w_{c}, w_{cz} equals between about 0.15-0.4 times w_{c}, w_{cz} equals between about 0.2-0.75 times w_{c}, w_{cz} equals between about 0.2-0.5 times w_{c}, or w_{cz} equals between about 0.2-0.4 times w_{c}.

As mentioned, width w_{cz} is a value in a range that has a minimum value and a maximum value. The maximum value, in one embodiment, is equal to or less than about 75% of the fluid flow channel width. In other embodiments, the maximum value is equal to or less than about 85%, 80%, 70<65%, 55%, 50%, 45%, 40%, 35%, 30%, 25% of the fluid flow channel width.

By way of example, imagine of fluid flow channel with a length of 17.70 mm and a width of 1.10 mm. If a reagent is used in the assay and the reagent is a solid with a dimension that needs to flow through the constriction zone, for example, a solid optically detectable particle with a diameter of between about 0.05-10 micrometers, then the minimum width of the channel in the constriction zone corresponds to the diameter of the particle. Alternatively, if there is not a solid reagent in the assay that needs to flow through the constriction zone, the minimum width of the channel in the constriction zone corresponds to a value that is equal to or greater than about 25% of the fluid flow channel width, or 25% of 1.10 mm in this imaginary example, which is 0.28 mm or greater. The maximum value for the width of the channel in the constriction zone, width w_{cz}, corresponds to equal to or less than about 75% of the fluid flow channel width, or 75% of 1.10 mm which is 0.825 mm or less. Thus, the minimum and maximum values in the range for width of the channel in the constriction zone for this imaginary channel is between 0.28 mm and 0.825 mm, inclusive.

The length of the channel in the constriction zone, lcz (FIG. 5B), in one embodiment, corresponds to a value in the range determined by (i) a minimum value that is equal to or greater than about 8% of the fluid flow channel length, and (ii) a maximum value that is equal to or less than about 75% of the fluid flow channel length. By way of example, and returning to the imaginary fluid flow channel mentioned in the preceding paragraph that has channel with a fluid flow channel length (lc) of 17.70 mm and a fluid flow channel width (wc) of 1.10 mm, the channel length in the constriction zone (lcz) is at least about 8% of 17.70 mm, which is 1.4 mm and is equal to or less than about 75% of 17.70 mm, which is 13.28 mm. Thus, the minimum and maximum values in the range for length of the channel in the constriction zone for this imaginary channel is between 1.4 mm and 13.28 mm, inclusive. In other embodiments, the minimum value in the range for the length of the channel in the constriction zone, lcz, is equal to or greater than about 2%, 3%, 4%, 5%, 7.5%, 9%, 10%, 12%, 15%, 18%, 20%, 22%, 25%, 28%, 30%, 32%, 35%, 40%, 45% or 50% of the fluid flow channel length. In other embodiments, the maximum value in the range for the length of the channel in the constriction zone, lcz, is equal to or less than about 95%, 90%, 85%, 80%, 70%, 65%, 60%, 55%, 50%, 45%, 40%, 35%, or 30% of the fluid flow channel length.

The dimensions and configuration of the channel in the constriction zone can vary along the channel length in the constriction zone, lcz. In some embodiments, the entrance and/or exit regions of the constriction zone are shaped to have a taper. The taper can be curved or angular. The taper, in an embodiment, extends from the fluid flow channel width to the channel constriction zone width.

In some embodiments, the channel entrance region of each fluid flow channel is configured to a fluid control feature. For example, and with reference to FIG. 4B, channel entrance region 412 of representative channel 408 can optionally comprise a fluid control feature. For example, in test strip 500 of FIGS. 5A-5B, a common, shared sample zone 502 is in direct fluid communication with an entrance region to each channel in the plurality of fluid flow channels, such as representative entrance region 504 to representative channel 506 (FIG. 5A). The entrance region 504 in this embodiment is a constriction region due to the diamond shaped, angular side walls of the fluid flow channel in this region. The entrance constriction region has a width w₁ (FIG. 5A) and a length L₁ (FIG. 5B), where in one embodiment, the entrance constriction region width w₁ is essentially the same as the width of the channel constriction zone w_{cz}. In one embodiment, the entrance constriction region width w₁ is between the minimum and maximum values discussed above for the width of the channel constriction zone w_{cz}. In one embodiment, the entrance constriction region width w₁ is a geometric shape that can be angular or non-angular. In one embodiment, the entrance constriction region comprises an angle that is between about 30-90°. In other embodiments, the entrance constriction region has a geometric shape of a half-rhombus, half-rectangle, half-square, quarter-square, quarter-rectangle, half-parallelogram, quarter-parallelogram, or half-kite.

As mentioned above with respect to FIG. 4B, a barrier region, (also referred to as a barrier extension region) is formed in the substrate to guide and control fluid placed in the sample zone into each channel in the plurality of fluid flow channels. FIG. 5B provides additional details on the barrier region, in accord with some embodiments. In test strip 500 of FIG. 5B, each fluid flow channel n (such as fluid flow channel 506 which is representative) has a length lc. Each fluid flow channel has opposing side walls, such as side walls 508, 510 of representative channel 506 in FIG. 5B. Each side wall has a width wₛ (FIG. 5B). In one embodiment, barrier region has a width w_{b} that is between about wₛ and about 10 wₛ, or between about wₛ and about 5 wₛ.

The number of channels in the plurality of fluid flow channels in a test assay can range from between 1-100, 1-50, 1-25, 1-20, 1-15, 2-100, 2-50, 2-25, 2-20, 2-15, 2-10, 3-100, 3-50, 3-25, 3-15, or 3-10, inclusive of any integer therein, for example, including but not limited to at least 2, 3, 4, 5, 6, 7, 8, 9, 10, 11, 12, 13, 14, 15, 16, 17, 18, 19 or 20 individual discrete fluid flow channels.

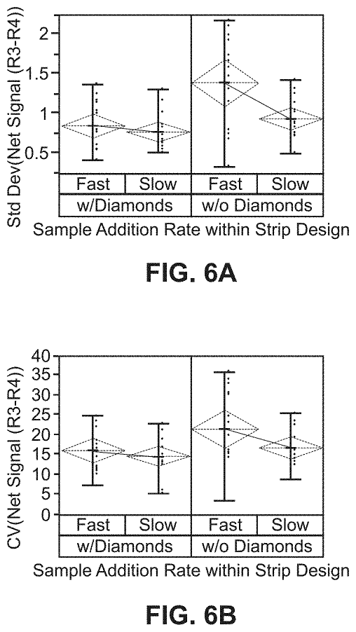

Test strips were prepared as described herein and tested to assess fluid flow rate and uniformity of fluid flow across the plurality of fluid flow channels. In one study, test strips essentially as depicted in FIGS. 5A-5B were prepared, with a constriction feature in the channel entrance region that was diamond shaped. Test strips without the diamond-shaped constriction feature were also prepared. A known volume of fluid was placed on the common sample zone quickly in one rapid deposit from a pipette or slowly over about 15 seconds. The rate of sample placement on the sample zone was varied to mimic how different users might deposit a sample onto the test device. FIGS. 6A-6B show the standard deviation (FIG. 6A) and coefficient of variation (FIG. 6B) from an analysis of the fluid flow on the test strips with and without the diamond-shaped fluid control feature in the fluid flow channel entrance region. FIG. 6A shows the standard deviation in the net signal for a fast sample addition rate and a slow sample addition rate, for test strips with and without the diamond-shaped constriction feature. FIG. 6A shows the coefficient of variability in the net signal for a fast sample addition rate and a slow sample addition rate, for test strips with and without the diamond-shaped constriction feature. Each of data points in the charts correspond to multiple (e.g., 20 or more) measurements with equal number of similarly designed test strips. It is seen that for embodiments with entrance constriction, the strip-to-strip variation is reduced, relative to embodiments with no entrance constriction.

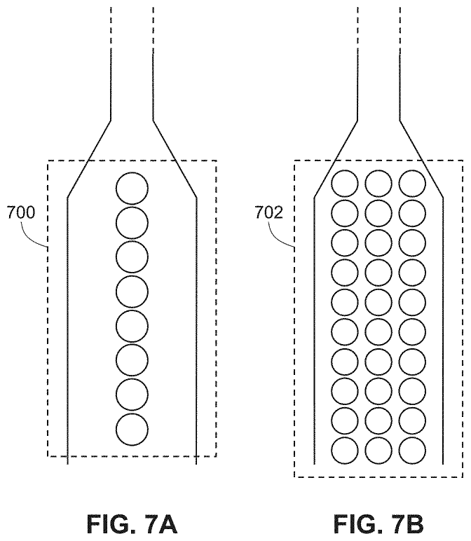

FIGS. 7A-B illustrate conjugate zones in a fluid flow channel, where the conjugate zone is an array of dots. Conjugate zone 700 of FIG. 7A is comprised of a 1×8 array of drops of a reagent that comprises mobilizable, detectable species. Conjugate zone 702 of FIG. 7B is comprised of a 3×10 array of drops of a reagent that comprises mobilizable, detectable species. It will be appreciated that each dot need not be the same composition, and that the capture zone of the fluid flow channel with immobilizable species may similarly be an array of drops of reagent. The reagent composition deposited to form a capture zone array or a conjugate zone array may include a mobilizable, detectable species or it may include a binding partner or species immobilized to the substrate or it may include a species useful as a control. The array, in one embodiment, includes m drops in one direction and n drops in a second direction, to form an m×n array, where m and/or n is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In one embodiment, n and m are the same, and in another embodiment, n and m are of a different value.

In another embodiment, the volume of formulation deposited on the substrate to form each drop 724 may be between about 20-1000 pL, or between about 50-800 pL, or between about 75-800 pL, or between about 100-600 pL, or between about 150-550 pL, or between about 200-500 pL, or between about 200-450 pL.

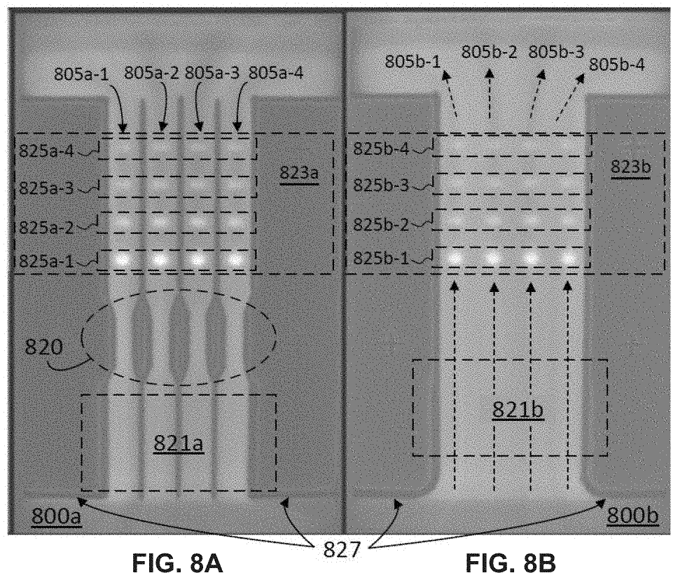

FIGS. 8A-8B are images of a test strip with a plurality of individual, discrete fluid flow channels (FIG. 8A) and one with a plurality of fluid flow channels that are not discrete (FIG. 8B) after testing for fluid flow. In FIG. 8A, an image of a test strip 800a with discrete, individual fluid channels 805a-1, 805a-2, 805a-3, and 805a-4 (hereinafter, collectively referred to as “fluid channels 805a”) is shown. FIG. 8B shows an image of a test strip 800b without discrete fluid flow channels—i.e., the fluid flow channels are not isolated and separated from one another over the length of the channel from its entrance at the common sample zone to its exit at a common exit region. Each of the test strips has a conjugate zone 821a, 821b, respectively. Hereinafter test strips 800a and 800b will be referred to, collectively, as “test strips 800.” Test strips 800 include a barrier wall 827. Test strip 800a includes a constriction region 820. In the capture zones, 823a, 823b, of each strip, the difference in performance can be seen. The test strip of FIG. 8A with the individual discrete channels and a constriction region yields bright and well resolved signal brightness in the capture zone drops 825a-1, 825a-2, 825a-3, and 825a-4 (hereinafter, collectively referred to as “drops 825a”). The signal resulting from test strip 800b is less bright and less resolved than that of test strip 800a. Further, fluid flow inhomogeneity is apparent from streaks 805b-1, 805b-2, 805b-3, and 805b-4 (hereinafter, collectively referred to as “streaks 805b”). Accordingly, in some embodiments, individual, discrete fluid channels in the plurality of channels is a feature that contributes to a higher and better resolved signal due to flow homogeneity.

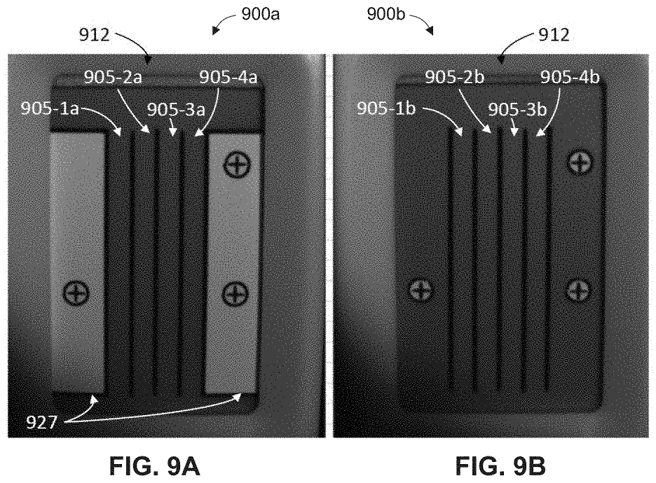

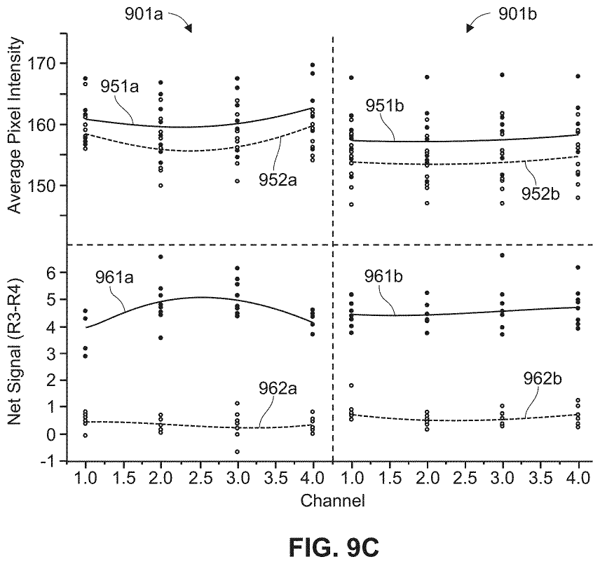

FIGS. 9A-9B illustrate a test strip 900a with a barrier wall 927 and a test strip 900b without a barrier wall. Test strips 900a and 900b will be referred to, collectively hereinafter, as “test strips 900.” Test strip 900a includes fluid channels 905-1a, 905-2a, 905-3a, and 905-4a (hereinafter, collectively referred to as “fluid channels 905a”). Likewise, test strip 900b includes fluid channels 905-1b, 905-2b, 905-3b, and 905-4b (hereinafter, collectively referred to as “fluid channels 905b”). These test strips were compared for performance by depositing a fluid sample in the sample zone and observing signal in the capture zone. FIG. 9C illustrates the results of the study on test strips 900. The results for test strip 900a are illustrated in the left panel (901a) of FIG. 9C, and the results for test strip 900b are illustrated in the right panel (901b). Curves 951a and 952a indicate signal and background measurements, respectively, for each of the channels 905a. Curves 951b and 952b indicate signal and background measurements for each of channels 905b.

Removal of the barrier wall 927 allows fluid sample deposited on the sample zone to flow towards absorbent pad 912 in test strip 900b. Initial raw signal intensity (curves 951a and 952a) show higher signal and background in outer channels for test strip 900a. Net signal 961a (obtained by subtracting curve 952a from curve 951a) results in a lower net signal on outer channels (e.g., channels 905-1a and 905-4a). A sample dependent bias may also be observed, in some embodiments. This may include a higher analyte concentration in the sample flow showing a larger background signal (e.g., curve 952a). Accordingly, test strip 900b with no barrier wall 927 may include a significant correction in channel bias (e.g., curves 951b, 952b, and 961b are more straight and horizontal).

Another study was conducted on test strips with and without a barrier wall. Test strips with four individual, discrete fluid flow channels were prepared, with one test strip having a barrier wall and one without a barrier wall. Signal from the capture zone was assessed after placing a fluid with a detectable species in the sample zone. The barrier wall was found to provide improved uniformity of fluid flow across the plurality of channels (data not shown).

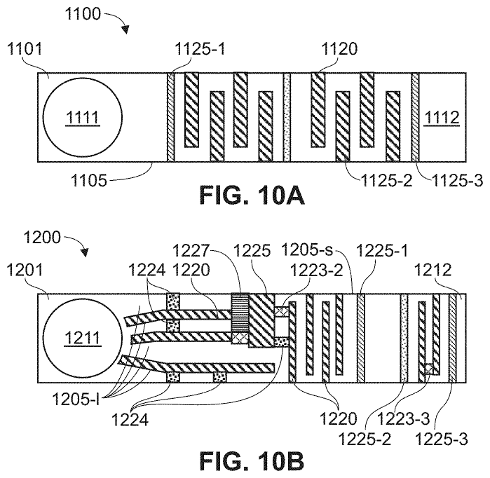

As mentioned above, the substrate of the test strips can be a laminate of a support member and a bibulous membrane. Also as discuss above, the bibulous membrane is processed or treated to etch away portions of the membrane to create the plurality of discrete, individual fluid flow channels and the fluid control features. Once the membrane is etched away, fluid traveling in the membrane contacts the support member. The hydrophobicity and hydrophilicity of the support member can be selected and optimized for control of the fluid. Moreover, the design of the fluid channels and the fluid control features can be varied to control rate of fluid flow. Some variations are shown in FIGS. 10A-10B. FIG. 10A illustrates a test strip 1100 having a substrate 1101 with a serpentine fluid channel 1105, according to some embodiments. Fluid channel 1105 is a flow reduction structure to induce more sample material to interact with a target capture zone 1125-2, thus increasing the sensitivity of detection of low positive results from the assay. The addition of a first control capture zone 1125-1 at the beginning of the assay, close to a sample pad 1111, and a second control capture zone 1125-3 at the end of the assay, close to absorbent pad 1112, provides accurate starting and finishing points for the test.

FIG. 10B illustrates a test strip 1200 including mixed longitudinal channels 1205-1 and serpentine channels 1205-s (hereinafter, collectively referred to as “fluid channels 1205”) with reagent patches 1224 and hydrophobic valves 1223-1, 1223-2, and 1223-3 (hereinafter, collectively referred to as “hydrophobic valves 1223”), according to some embodiments. A hydrophilic mixing zone 1227 may be disposed between a longitudinal channel 1205-1 and a serpentine channel 1205-s, to regulate the speed of sample flow between a sample pad 1211 and a test capture zone 1225. Moreover, in some embodiments, hydrophilic mixing zone 1227 may be adjacent to a hydrophobic valve 1223-1, each having a pre-selected dimension (e.g., width and length) to obtain a desired fluid flow rate along fluid channels 1205. Die cuts 1220 (or hydrophobic valves 1223) may be used to create gated areas where the flow rate of the assay is slowed down, or throttled to allow a reagent patch 1224 to interact with the sample flow. A narrower gate will reduce sample flow. In some embodiments, the throttling of one fluid channel 1205 may allow a slower fluid channel to move forward, so that all fluid channels and all reagents reach test capture zone 1225-2 more or less simultaneously, or at approximately the same time.

Embodiments consistent with test strip 1200 may include different combinations of longitudinal fluid channels 1205-1, and serpentine fluid channels 1205-s, with hydrophobic 1223 and hydrophilic 1227 gates, depending on the affinity of different reagents 1224 with the respective target analytes in the sample fluid. Accordingly, the selection of the shape and distribution of the different flow components illustrated in test strip 1200 may vary according to a desire for obtaining a rapid, yet homogenous (e.g., approximately simultaneous) response to the different components of the assay at test capture zone 1225-2. In some embodiments, this is desirable so as to have a single ending point of the assay test, which simplifies the measurement and analysis logistics.

Similarly to test strip 1100 (cf. FIG. 10A), a first control capture zone 1225-1 at the beginning of fluid channel 1205-s, proximal to sample pad 1211, and a second control capture zone 1225-3 at the end of the assay, close to absorbent pad 1212, provides accurate starting and finishing points for the test. In some embodiments, a hydrophobic valve 1223-3 may be positioned in fluid channel 1205-s to slow the ending of the assay, and ensure that test capture zone 1225-2 sufficiently interacts with the sample fluid before the ending of the assay.

Hydrophobic valves 1223, hydrophilic mixture zone 1227, and test capture zone 1225 are fluid features included in substrate 1201 and have a shape and size to inhibit or enhance sample flow across test strip 1200, as desired. In some embodiments, the details of fluid features in test strip 1200 are selected to provide time at certain stages in the assay (e.g., to allow for reaction with one of reagents 1224 to occur, or to allow for a conjugate immobilization in test capture zone 1225-2 to complete). In some embodiments, the fluid features in test strip 1200 may be selected to steer flow into separate capture zones 1225 arranged in an array matrix. The capabilities of the fluid features in test strip 1200 may be fully exploited by a digital capture device (e.g., as in image-capturing device 130, cf FIG. 1), as disclosed herein.

### Methods of Use

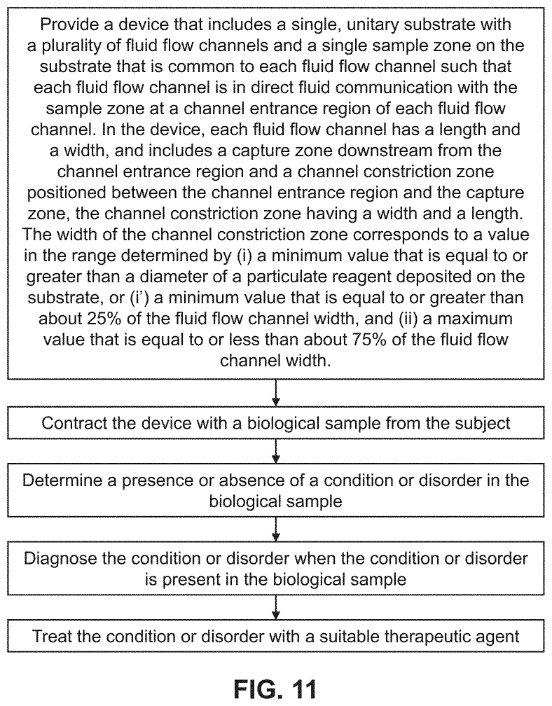

FIG. 11 is a flow chart illustrating steps in a method 1300 for remotely diagnosing a disease with an image-capturing device, according to some embodiments. Method 1300 may be performed at least partially by a computer or an image-capturing device as in the architecture illustrated in FIG. 1. Accordingly, at least some of the steps in method 1300 may be performed by a processor executing instructions stored in a memory. Further, methods consistent with the present disclosure may include at least one step as described in method 1300. In some embodiments, methods consistent with the present disclosure include one or more steps in method 1300 performed in a different order, simultaneously, almost simultaneously, or overlapping in time.

Step 1302 includes providing a device that includes a single, unitary substrate with a plurality of fluid flow channels and a single sample zone on the substrate that is common to each fluid flow channel such that each fluid flow channel is in direct fluid communication with the sample zone at a channel entrance region of each fluid flow channel. In the device, each fluid flow channel has a length and a width, and includes a capture zone downstream from the channel entrance region and a channel constriction zone positioned between the channel entrance region and the capture zone, the channel constriction zone having a width and a length. The width of the channel constriction zone corresponds to a value in the range determined by (i) a minimum value that is equal or greater than a diameter of a particular reagent deposited on the substrate, or (i′) a minimum value that is equal to or greater than about 25% of the fluid flow channel width, and (ii) a maximum value that is equal to or less than about 75% of the fluid flow channel width.

Step 1304 includes contacting the device with a biological sample from the subject.

Step 1306 includes determining a presence or absence of a condition or disorder in the biological sample. In some embodiments, step 1306 includes determining a presence or absence of a bacterial infection, a viral infection, or an addiction or misuse of a drug. In some embodiments, step 1306 includes determining a presence or absence of a viral infection that includes a respiratory infection. In some embodiments, step 1306 includes determining a presence or absence of a bacterial infection that includes Lyme disease or sepsis.

Step 1308 includes diagnosing the condition or disorder when the condition or disorder is present in the biological sample.

Step 1310 includes treating the condition or disorder with a suitable therapeutic agent. In some embodiments, step 1310 includes treating the condition or disorder with an antibiotic.

### Hardware Overview

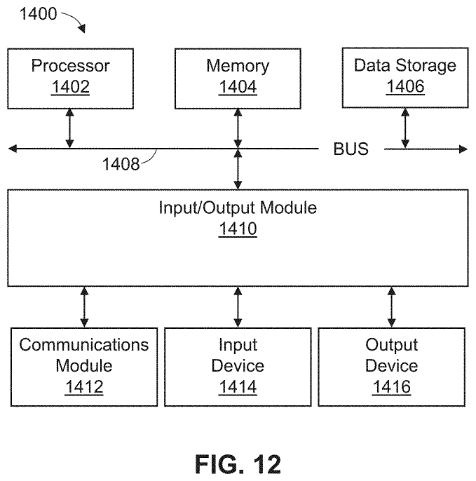

FIG. 12 is a block diagram illustrating an example computer system 1400 with which the image-capturing device and the server of FIG. 1, and the methods disclosed herein (e.g., method 1300, cf FIG. 11) can be implemented, according to some embodiments. In certain aspects, computer system 1400 may be implemented using hardware or a combination of software and hardware, either in a dedicated server, or integrated into another entity, or distributed across multiple entities.

Computer system 1400 (e.g., server 110, image-capturing device 130) includes a bus 1408 or other communication mechanism for communicating information, and a processor 1402 coupled with bus 1408 for processing information. By way of example, computer system 1400 may be implemented with one or more processors. Processor 1402 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 1400 can include, in addition to hardware, code that creates an execution environment for the computer program in question, e.g., code that constitutes processor firmware, a protocol stack, a database management system, an operating system, or a combination of one or more of them stored in an included memory 1404, such as a Random Access Memory (RAM), a flash memory, a Read-Only Memory (ROM), a Programmable Read-Only Memory (PROM), an Erasable PROM (EPROM), registers, a hard disk, a removable disk, a CD-ROM, a DVD, or any other suitable storage device, coupled to the bus for storing information and instructions to be executed by processor 1402. Processor 1402 and memory 1404 can be supplemented by, or incorporated in, special purpose logic circuitry.

The instructions may be stored in memory 1404 and implemented in one or more computer program products, i.e., one or more modules of computer program instructions encoded on a computer-readable medium for execution by, or to control the operation of, computer system 1400, and according to any method well-known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command-line interface languages, compiled languages, concurrent languages, curly-bracket languages, dataflow languages, data-structured languages, declarative languages, esoteric languages, extension languages, fourth-generation languages, functional languages, interactive-mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. The memory may also be used for storing temporary variable or other intermediate information during execution of instructions to be executed by the processor.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors 1402 executing one or more computer programs to perform functions by operating on input data and generating output.

Computer system 1400 further includes a data storage device 1406 such as a magnetic disk or optical disk, coupled to the bus for storing information and instructions. Computer system 1400 may be coupled via an input/output module 1410 to various devices. Input/output module 1410 can be any input/output module. Exemplary input/output modules include data ports such as USB ports. Input/output module 1410 may be configured to connect to a communications module. Exemplary communications modules include networking interface cards, such as Ethernet cards and modems. In certain aspects, input/output module 1410 may be configured to connect to a plurality of devices, such as an input device 1414 and/or an output device 1416. Exemplary input devices 1414 include a keyboard and a pointing device, e.g., a mouse or a trackball, by which a user can provide input to the computer system. Other kinds of input devices 1414 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback; and input from the user can be received in any form, including acoustic, speech, tactile, or brain wave input. Exemplary output devices 1416 include display devices, such as an LCD (liquid crystal display) monitor for displaying information to the user.

In some embodiments, computer system 1400 is a network-based, voice-activated device accessed by the user. Input/output devices 1414 and 1416 may include a microphone providing the queries in voice format, and receiving multiple inputs from the user also in a voice format, in the language of the user. Further, in some embodiments, a neural linguistic algorithm may cause the voice-activated device to contact the user back and receive a user selection of the respiratory mask via a voice command or request.

According to one aspect of the present disclosure, image-capturing device 130 and server 110 can be implemented using computer system 1400 in response to processor 1402 executing one or more sequences of one or more instructions contained in memory 1404. Such instructions may be read into memory 1404 from another machine-readable medium, such as data storage device 1406. Execution of the sequences of instructions contained in the main memory causes processor 1402 to perform the process steps described herein. One or more processors in a multi-processing arrangement may also be employed to execute the sequences of instructions contained in the memory. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system 1400 that includes a back-end component, e.g., as a data server, or that includes a middleware component, e.g., an application server, or that includes a front-end component, e.g., image-capturing device 130 having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. The communication network (e.g., network 150) can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system 1400 can include image-capturing devices and servers wherein the image-capturing device and server are generally remote from each other and typically interact through a communication network (e.g., image-capturing device 130, server 110, and network 150, cf. FIG. 1). The relationship of image-capturing device and server arises by virtue of computer programs running on the respective computers and having an image-capturing device-server relationship to each other. The computer system can be, for example, and without limitation, a desktop computer, laptop computer, or tablet computer. The computer system can also be embedded in another device, for example, and without limitation, a mobile telephone, a PDA, a mobile audio player, a Global Positioning System (GPS) receiver, a video game console, and/or a television set top box.

The term “machine-readable storage medium” or “computer-readable medium” as used herein refers to any medium or media that participates in providing instructions to the processor for execution. Such a medium may take many forms, including, but not limited to, non-volatile media, volatile media, and transmission media. Non-volatile media include, for example, optical or magnetic disks, such as the data storage device. Volatile media include dynamic memory, such as the memory. Transmission media include coaxial cables, copper wire, and fiber optics, including the wires that include the bus. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter effecting a machine-readable propagated signal, or a combination of one or more of them.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

To the extent that the term “include,” “have,” or the like is used in the description or the claims, such term is intended to be inclusive in a manner similar to the term “include” as “include” is interpreted when employed as a transitional word in a claim. The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products. Other variations are within the scope of the following claims.

In one aspect, a method may be an operation, an instruction, or a function and vice versa. In one aspect, a claim may be amended to include some or all of the words (e.g., instructions, operations, functions, or components) recited in other one or more claims, one or more words, one or more sentences, one or more phrases, one or more paragraphs, and/or one or more claims.

To illustrate the interchangeability of hardware and software, items such as the various illustrative blocks, modules, components, methods, operations, instructions, and algorithms have been described generally in terms of their functionality. Whether such functionality is implemented as hardware, software, or a combination of hardware and software depends upon the particular application and design constraints imposed on the overall system. Skilled artisans may implement the described functionality in varying ways for each particular application.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for.”

While this specification contains many specifics, these should not be construed as limitations on the scope of what may be described, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially described as such, one or more features from a described combination can in some cases be excised from the combination, and the described combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the described subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately described subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

It is claimed:

1. A device, comprising:
a single, unitary substrate comprising a plurality of discrete fluid flow channels,
a single sample zone on the substrate that is common to each fluid flow channel such that each fluid flow channel is in direct fluid communication with the sample zone at a channel entrance region of each fluid flow channel;
each fluid flow channel having a length and a width, each fluid flow channel comprising a capture zone downstream from the channel entrance region and a channel constriction zone positioned between the channel entrance region and the capture zone, the channel constriction zone having a width and a length, the channel constriction zone width corresponding to a value in the range determined by (i) a minimum value that is equal to or greater than a diameter of a particulate reagent deposited on or to be deposited on the substrate or (i′) a minimum value that is equal to or greater than about 25% of the fluid flow channel width, and (ii) a maximum value that is equal to or less than about 75% of the fluid flow channel width.

2. The device of claim 1, wherein the channel constriction zone length corresponds to a value in the range determined by (i) a minimum value that is equal to or greater than about 8% of the fluid flow channel length, and (ii) a maximum value that is equal to or less than about 75% of the fluid flow channel length.

3. The device of claim 1, wherein the channel constriction zone length corresponds to a value in the range determined by (i) a minimum value that is equal to or greater than about 10% of the fluid flow channel length, and (ii) a maximum value that is equal to or less than about 65% of the fluid flow channel length.

4. The device of claim 1, wherein the channel constriction zone width is variable along the channel constriction zone length.

5. The device of claim 4, wherein the channel constriction zone includes a taper region at an entrance region into the channel constriction zone or at an exit region of the channel constriction zone.

6. The device of claim 5, wherein the taper region extends from the fluid flow channel width to the channel constriction zone width.

7. The device of claim 1, wherein the channel constriction zone is non-angular along its length.

8. The device of claim 1, wherein the particulate reagent is an optically detectable solid particle.

9. The device of claim 8, wherein the solid particle is a fluorescent particle having a diameter of between about 0.05-750 microns (0.00005-0.75 mm).

10. The device of claim 8, wherein the solid particle is a fluorescent particle having a diameter of between about 0.05-10 microns (0.00005-0.01 mm).

11. The device of claim 1, further comprising an entrance constriction region in each fluid flow channel, the entrance constriction region positioned between the common sample zone and the channel constriction zone.

12. The device of claim 11, wherein the entrance constriction region is positioned at the channel entrance region.

13. The device of claim 11, wherein the entrance constriction region has a width and a length, wherein the entrance constriction region width is essentially the same as the channel constriction zone.

14. The device of claim 11, wherein the entrance constriction region is angular.

15. The device of claim 14, wherein the entrance constriction region comprises an angle that is between about 30-90°.

16. The device of claim 14, wherein the entrance constriction region has a geometric shape of a half-rhombus, half-rectangle, half-square, quarter-square, quarter-rectangle, half-parallelogram, quarter-parallelogram, or half-kite.

17. The device of claim 11, wherein the entrance constriction region is non-angular.

18. The device of claim 1, wherein the plurality of discrete fluid flow channels comprises n fluid flow channels, where n is between 2-20.

19. The device of claim 18, wherein each fluid flow channel n is identified by an integer between 1 and n, and wherein fluid flow channel l and fluid flow channel n each comprise an outer channel wall with a width w and a barrier extension region positioned at the channel entrance region with a width of between about w and about 10 w.

20. The device of claim 19, wherein the barrier extension region has a width between about w and about 5 w.

21. The device of claim 1, wherein the substrate is nitrocellulose.

22. The device of claim 1, wherein each capture zone comprises a different capture reagent.

23. The device of claim 22, wherein each capture zone comprises a capture reagent for an infectious agent.

24. The device of claim 23, wherein the infectious agent is selected from respiratory syncytial virus, influenza A virus, influenza B virus, and human metapneumovirus.

25. The device of claim 23, wherein the infectious agent is a Borrelia species.

26. The device of claim 23, wherein each capture zone comprises a capture reagent for a drug of abuse.

27. The device of claim 26, wherein the drug of abuse is selected from fentanyl, buprenorphine, oxycodone, and 7-aminoclonazepam.

28. The device of claim 22, wherein each capture zone comprises a capture reagent to discriminate bacterial from viral infection.

29. The device of claim 28, wherein the capture reagent comprises a reagent that binds or interacts with tumor necrosis factor-related apoptosis-inducing ligand (TRAIL), C-reactive protein (CRP), interferon-gamma-induced protein-10 (IP-10), Radical S-Adenosyl Methionine Domain Containing 2 (RSAD2), MX dynamin like GTPase 1 (MX1 or MxA), MX dynamin like GTPase 2 (MX2 or MxB), neutrophil gelatinase-associated lipocalin (NGAL), and procalcitonin (PCT).

30. The device of claim 22, wherein the capture reagent is a (i) monoclonal or a polyclonal antibody, (ii) a fragment of TRAIL, CRP, IL-10, RSAD2, MX1, MX2, NGAL, PCT or (iii) a fragment of an infectious agent.

31. The device of claim 1, wherein the substrate is a laminate comprising a hydrophobic material.

32. A method of diagnosing, treating, or both a condition or disorder in a subject, comprising:
providing a device of any preceding claim,
contacting the device with a biological sample from the subject; and
determining presence or absence of the condition or disorder, and
optionally, diagnosing the condition or disorder, and
optionally treating the condition or disorder with a suitable therapeutic agent.

33. The method of claim 32, wherein the condition or disorder is a bacterial infection, a viral infection, or an addiction or misuse of a drug.

34. The method of claim 33, wherein the viral infection is a respiratory infection.

35. The method of claim 33, wherein the bacterial infection is Lyme disease or sepsis.

36. The method of any one of claims 32, wherein treating the condition or disorder with a suitable therapeutic agent comprises treating with an antibiotic.
